(19) United States
(12) Patent Application Publication
(10) Pub. No.: US 2017/0246129 A1
(43) Pub. Date: Aug. 31, 2017

(54) COMPOSITION USING GUANIDINE COMPOUND FOR SUPPRESSING TRANSFORMING GROWTH FACTOR (TGF)-beta

(71) Applicants: Peking University Third Hospital, Beijing (CN); Institute of High Energy Physics, Chinese Academy Sciences, Beijing City (CN); Institute of Chemistry, Chinese Academy of Sciences, Beijing City (CN)

(72) Inventors: Youyi ZHANG, Beijing (CN); Han XIAO, Beijing (CN); Jingyuan LI, Beijing (CN); Xiaohong FANG, Beijing (CN); Zhonghe XU, Beijing (CN); Zhizhen LU, Beijing (CN); Mingliang ZHANG, Beijing (CN)

(21) Appl. No.: 15/054,450

(22) Filed: Feb. 26, 2016

(51) Int. Cl. A61K 31/155

(52) U.S. Cl. CPC A61K 31/155

(57) ABSTRACT

The present invention relates to a composition as a transforming growth factor (TGF)-β suppressor which comprises an effective amount of guanidine compound or a pharmaceutically acceptable salt. More particularly, the present invention relates to a composition comprising a guanidine compound or a pharmaceutically acceptable salt thereof as a TGF-β suppressor, wherein the composition is characterized by suppression or reduction of TGF-β activity which is a cause of disease. The present invention relates to a method of treating various TGF-β associated diseases, the method comprising administering to the subject a composition comprising a guanidine compound or a pharmaceutically acceptable salt thereof as a TGF-β suppressor which can prevent or treat TGF-β associated diseases by suppressing or reducing TGF-β activity.

### FIELD OF THE INVENTION

The present invention is related to a new application for a guanidine compound, especially as a suppressor for transforming growth factor (TGF)-β. The present invention provides a method using a composition comprising a guanidine compound or a pharmaceutically acceptable salt thereof as a TGF-β suppressor to prevent or treat TGF-β associated diseases.

### BACKGROUND OF INVENTION

Transforming growth factor β (TGF-β1) is a prototypic member of a large superfamily of secreted proteins that include three TGF-β isoforms (TGF-β1, TGF-β2 and TGF-β3), activins, growth and differentiation factors, bone morphogenetic proteins (BMPs), inhibins, nodal, and anti-Mullerian hormone. TGF-β1 is a pleiotropic cytokine that controls proliferation, differentiation, embryonic development, angiogenesis, wound healing and other functions in many cell types. TGF-β1 is involved in the progression of many diseases, including cancer and fibrotic, cardiovascular, and immunological diseases. It is associated with fibrosis, epithelial-to-mesenchymal transition (EMT) and inflammation which are important pathological changes in the aforementioned diseases.

The TGF-β1 signaling pathway requires two cell-surface serine/threonine kinase receptors, type II (TβRII) and type I (TβRI) TGF-β1 receptors. In general, TGF-β1 binds to TβRII, which recruits TβRI to form a heteromeric complex of TβRI-TβRII. The formation of this complex leads to the phosphorylation of TβRI and subsequent phosphorylation of receptor-regulated Smads (R-Smads, i.e., Smad2, Smad3, etc), which bind a co-Smad (Smad4). The R-Smad/co-Smad complexes translocate and accumulate in the nucleus and subsequently regulate target genes. (Moustakas, A. & Heldin, C. H. Development. 136, 3699-3714 (2009)).

Augmented TGF-β expression results in tumor progression and metastasis. The mechanisms including TGF-β induced EMT, evasion of the immune system, and promotion of cancer cell proliferation by modulation of the tumor microenviroment. Fibrosis is another important pathological changes induced by TGFβ. TGFβ exacerbates fibrosis through increasing extracellular matrix synthesis and deposition. TGFβ1-induced EMT also plays a key role in organ fibrosis.

In addition to fibrosis and tumors, TGF-β is involved in numerous other diseases. Thus, targeting the TGFβ signaling pathway has become attractive for drug development. Currently, therapeutic strategies against the TGF-β family include three approaches: 1) inhibition at the translational level using antisense oligonucleotides, 2) inhibition of the ligand-receptor interaction using ligand traps and anti-receptor monoclonal antibodies, and 3) inhibition of the receptor-mediated signaling cascade using inhibitors and aptamers of TGF-β receptor kinases Only few drugs have been developed through preclinical to clinical trials and many more have been tested only in preclinical systems. These approaches also have their specific challenges to limit their application. The antisense oligonucleotides have limited ability to reach targeted tissue. Although monoclonal antibodies have advantages of specificity and efficacy, they also need to overcome significant physical barriers to penetrate targeted tissue and have extremely high production costs. The inhibitors of TGF-β receptor kinases have side effects by possible cross-inhibition of other kinases. (Akhurst R J, Hata A. Nat Rev Drug Discov 2012;11:790-811; Nagaraj N S, Datta P K. Expert Opin Investig Drugs 2010;19:77-91).

Metformin was originally derived from the French lilac Galega officinalis, and it is currently a widely prescribed biguanide used as a first-line antidiabetic drug. Metformin is safe and effective in the treatment of diabetes and does not induce hypoglycemia. Beyond its known blood glucose lowering effects, metformin has been shown to elicit beneficial effects on cardiovascular diseases, polycystic ovary syndrome, diabetic nephropathy, and cancer.

However, the mechanisms underlying the pleiotropic effects of metformin remain elusive. Our previous study revealed that metformin inhibits cardiac fibrosis by inhibiting the TGF-β1-Smad3 signaling pathway (Xiao, H. et al. Cardiovasc. Res. 87, 504-513 2010). To date, there is no study report saying that metformin or a guanidine compound can be applied as a TGF-β suppressor to treat or prevent TGF-β associated diseases in which TGF-β1 signaling malfunctions are indicated.

### SUMMARY OF INVENTION

One object of the present invention is to provide a novel safe TGF-β suppressor.

Another object of the present invention is to provide a novel application for guanidine compound or a pharmaceutically acceptable salt thereof as a TGF-β suppressor.

Another object of the present invention is to provide a composition which comprises a guanidine compound or a pharmaceutically acceptable salt thereof.

Another object of the present invention is to provide a therapeutic method for TGF-β associated diseases by blocking the binding of TGF-β ligand and its receptors.

The present invention identified guanidine compound or a pharmaceutically acceptable salt thereof as a TGF-β suppressor through interacting with the TGF-β1 ligand, thereby blocking the binding of TGF-β1 to TβRII and resulting in decreased downstream signaling.

In this regard, the present invention demonstrated that metformin inhibited [125I]-TGF-β1 binding to its receptor in 3T3 mouse fibroblasts. Single molecule fluorescence imaging showed that metformin inhibited type II TGF-β1 receptor dimerization which is essential for downstream signaling transduction. Using single-molecule force spectroscopy, metformin was found to reduce the binding probability but not binding force of TGF-β1 to its type II receptor. Furthermore, molecular docking and molecular dynamics simulations suggested that metformin interacts with TGF-β1 to block the binding to its receptor, and thereby antagonizes TGF-β1 effects. Surface plasmon resonance based assay confirmed the binding of metformin and TGF-β1. Western blotting analysis suggested that some other guanidine compounds which have similar structure with metformin also inhibit TGF-β1 downstream signaling and can be applied as TGF-β1 suppressor.

Thereby, in one general aspect, the present invention provided a novel safe TGF-β suppressor which comprises an effective amount of guanidine compounds or a pharmaceutically acceptable salt thereof, wherein the guanidine compound is selected from the group consisting of metformin, phenylbiguanide, 1,3-Di(o-tolyl)guanidine, and buformin.

In some embodiments of the present invention, the pharmaceutically acceptable salt is hydrochloride. Namely, pharmaceutically acceptable salt is metformin hydrochloride and buformin hydrochloride.

In some embodiments of the present invention, the present invention provided a novel application for guanidine compound or a pharmaceutically acceptable salt thereof as a TGF-β suppressor.

In some embodiments of the present invention, the TGF-β suppressor composition of the present invention further comprises a pharmaceutically acceptable carrier or excipient.

In some embodiments of the present invention, wherein the composition can prevent or treat TGF-β associated diseases.

In some embodiments of the present invention, the TGF-β associated diseases includes but not limits to fibrosis, cancer, myelodysplastic syndrome, scleroderma, restenosis following coronary artery bypass and angio-plasty, marfan syndrome, postoperative scarring.

In some embodiments of the present invention, metformin compound or a pharmaceutically acceptable salt is applied as a TGF-β suppressor to prepare associated medicine.

In another general aspect, the present invention provided a novel method of treating TGF-β associated diseases, the method comprising administering to the subject metformin compound or a pharmaceutically acceptable salt, which interacts with TGF-β to block the binding to its receptor and thereafter suppress TGF-β activity to treat TGF-β associated diseases.

In some embodiments of the present invention, the pharmaceutically acceptable salt is hydrochloride.

In some embodiments of the present invention, the TGF-β associated diseases includes but not limits to fibrosis, cancer, myelodysplastic syndrome, scleroderma, restenosis following coronary artery bypass and angio-plasty, marfan syndrome, postoperative scarring.

In some embodiments of the present invention, the effective amount of metformin compound or a pharmaceutically acceptable salt thereof comprises about 850 mg/day to about 2000 mg/day.

In the present invention, we demonstrated that metformin antagonized TGF-β1 signaling by interacting with TGF-β1 ligand to block the binding to its receptor TβRII, then inhibiting the receptor dimerization and the subsequent signaling pathway. Metformin is not metabolized and is excreted unchanged in the urine. Also, the plasma protein binding of metformin is negligible. Blood or plasma metformin concentrations are usually in a range of 1-4 μg/mL (about 6-24 μM) in persons receiving the drug therapeutically (Glucophage (metformin hydrochloride tablets) Label Information [article online], 2008.). Our results showed that the K_{D} value for the binding of metformin and TGF-β1 is 15.9 μM. It is conceivably thought that metformin antagonizes TGF-β1 with its original structure and therapeutically blood concentration in vivo.

It is generally accepted that metformin acts via the activation of AMP-activated protein kinase and the inhibition of mitochondrial respiratory-chain complex. Here, we discovered that metformin antagonizes TGF-β1 signaling by directly binding to TGF-β1. Consistent with the well-established role of TGF-β1 in the exacerbation of fibrosis, our previous study and other studies have shown that metformin treatment attenuates cardiac fibrosis, liver fibrosis and renal fibrosis(Xiao, H. et al. Cardiovasc Res. 87, 504-513 2010). In addition, metformin has been shown to inhibit TGF-(31-induced epithelial-to-mesenchymal transition which plays a key role in carcinoma progression and organ fibrosis (Cufi, S. et al. Cell Cycle 2010; 9:4461-4468). Moreover, clinical trials have suggested that metformin is associated with decreased cancer risk and improved prognosis in cancer patients (Rizos, C. V. & Elisaf, M. S. Eur J Pharmacol 2013; 705:96-108). These findings support the idea that metformin exerts a protective effect against organ fibrosis and malignant tumor progression by blocking TGF-β1.

In addition to fibrosis and tumors, TGF-β is involved in numerous other diseases. Thus, targeting the TGF-β signaling pathway has become attractive for drug development. Currently, therapeutic strategies against the TGF-β family include three approaches: 1) inhibition at the translational level using antisense oligonucleotides, 2) inhibition of the ligand-receptor interaction using ligand traps and anti-receptor monoclonal antibodies, and 3) inhibition of the receptor-mediated signaling cascade using inhibitors and aptamers of TGF-β receptor kinases. However, these approaches have specific challenges that limit their application, such as the limited ability of an antisense oligonucleotides and monoclonal antibodies to reach the targeted tissue (Akhurst R J, Hata A. Nat Rev Drug Discov 2012; 11:790-811; Nagaraj N S, Datta P K. Expert Opin Investig Drugs 2010; 19:77-91). In contrast, metformin is a small molecule compound that can easily reach the targeted tissue. Inhibitors of TGF-β receptor kinases have side effects that occur through the potential cross-inhibition of other kinases. Conversely, metformin has been shown to be safe and have fewer side effects over decades of use. In addition, metformin has beneficial effects beyond targeting TGF-β and based on the interaction mode between metformin and TGF-β, additional compounds can be developed to target TGF-β with higher specificity and potency.

In summary, the present invention identified guanidine compounds, especially metformin as a novel TGF-β1 suppressor, and this action underlies the pleiotropic effects of the drug. This finding strongly supports the clinical use of metformin as a treatment for numerous diseases beyond diabetes where TGF-β1 signaling malfunctions are indicated. In addition, the present invention provides insights that can be used in the development of new compounds targeting TGF-β1.

### DESCRIPTION OF DRAWINGS

FIG. 1 Metformin inhibited [125I]-TGF-β1 receptor binding to 3T3 mouse fibroblasts. The results are expressed as the percentage of specific binding in the absence of metformin (n=4).

FIGS. 2A-2D (A) Typical single-molecule image of TβRII-GFP on the HeLa cell membrane. After transfection with TβRII-GFP for 4 h, HeLa cells were imaged using total internal reflection fluorescence microscopy (TIRFM). The diffraction-limited spots (5 * 5 pixel regions) enclosed with green circles represent the signals from individual TβRII-GFP molecules, and they were chosen for the intensity analysis. Scale bar, 5 μm. (B) and (C) Two representative time course graphs of GFP emissions after background correction demonstrating one- and two-step bleaching, respectively. The arrows indicate the bleaching steps. The individual TβRII-GFP molecules were monomers when they were bleached in one step (B) and dimers when they were bleached in two steps (C). (D) Metformin inhibited TGF-β1-induced TβRII dimerization as shown by single-molecule imaging. Fraction of two-step bleaching events for TβRII-GFP molecules (counted spots were set as 100%) was represented as the dimer percentage. Prior to single-molecule fluorescence imaging, metformin and TGF-β1 (10 ng/mL) were premixed for 2 h, and HeLa cells were then treated with the mixture for 15 min at 37° C. The data were presented as the mean±SEM. The data were presented as the mean±SEM (n=5-16). ANOVA combined with Tukey's post-hoc test was used. *P<0.05 vs. control group, #P<0.05 vs. TGF-β1 group.

FIG. 3 The schematic diagram of TGF-β1-TβRII binding force measurements which were obtained with TGF-β1-modified atomic force microscopy (AFM) tips on HeLa cells.

FIG. 4A-D Metformin inhibits the binding probability but not binding force of TGF-β1 and TβRII. TGF-β1-TβRII binding force measurements were obtained with TGF-β1-modified atomic force microscopy (AFM) tips on HeLa cells, which express TβRII. (A) Histograms of binding forces of TGF-β1/TβRII in the untreated cells and (B) the cells treated with 50 μM metformin. (C) Binding forces of TGF-β1/TβRII in cells treated with/without 50 μM metformin. (D) Binding probability of TGF-β1 and TβRII when the cells were treated with metformin and/or the blocking reagent (anti-TGF-β1 antibody). Blocking experiments were performed by the addition of free TGF-β1 monoclonal antibodies into the solution. Data were expressed as the mean±SEM from 3 independent experiments. *P<0.05.

FIG. 5A-B Molecular docking for TβRII:metformin and TGF-β1:metformin (A) Structure of the TGF-β1: metformin complex after relaxation. The surface region of TβRII recognized by TGF-β1 is shown in in dark grey. (B) Structure of the TβRII: metformin complex after relaxation. The surface region of TβRII recognized by TGF-β1 is shown in in dark grey.

FIG. 6A-B Molecular dynamics simulations for TβRII:metformin and TGF-β1:metformin (A) Root-mean-square deviation (RMSD) of TGF-β1 and metformin relative to TGF-β1 during the last 25-ns trajectory. (B) RMSD of TβRII and metformin relative to TβRII.

FIG. 7A Binding site of metformin consists of the β-sheet1 and β-sheet2 of TGF-β1.

FIG. 7B Residues in direct contact with metformin (depicted using the LIGPLOT program with a cutoff of 3.9 Å).

FIG. 8 Sensorgrams for the binding of metformin and TGF-β1. TGF-β1 was covalently coupled to a CM5 chip, and metformin was injected in a two-fold dilution concentration series ranging from 62.5 μM to 1.9 μM. The steady-state values were calculated from the sensorgrams and plotted against the concentrations. The data were fit to a single-site binding model to calculate the K_{D}.

FIG. 9 The effects of other compounds which have similar structure with metformin on TGF-β1 induced phosphorylated-Smad3. Different drugs and TGF-β1 (5 ng/mL) were separately premixed for 2 h and then treat cells for 30 minutes. Western blot analysis of phosphorylated-Smad3 (β-Smad3), Smad3, and GAPDH in the cells were performed.

### DETAILED DESCRIPTION OF THE INVENTION

The following detailed description of invention will be better understood when read in conjunction with the appended drawings. It should be understood, however, that the invention is not limited to the precise arrangements and instrumentalities shown. All the procedures which are not described in details are performed according to the routine operation or the manufacturer's instructions.

All the reagents in the following embodiments are commercial available. Metformin applied in the following embodiments is metformin hydrochloride.

### [¹²⁵I]-TGFβ1 Binding Assay

3T3 mouse fibroblasts were seeded in a 24 well-plate and cultured in DMEM supplemented with 10% FBS and antibiotics (100 U/mL penicillin-streptomycin). When cells were at a near-confluent stage, 50 pM [¹²⁵I]-TGFβ1 with or without different concentrations of metformin were added. After 4 h at 4° C., the medium was removed and cells were washed five times with ice-cold binding buffer (50 mM HEPES, 128 mM NaCl, 5 mM KCl, 5 mM MgSO₄, and 1.2 mM CaCl₂). The cells were then solubilized using binding buffer containing 1% Triton X-100 and the radioactivity was measured. Non-specific binding was determined in the presence of an excess (10 nM) of unlabeled TGFβ1. The IC₅₀ value of [¹²⁵I]-TGFβ1 binding inhibition by metformin was determined in four independent experiments.

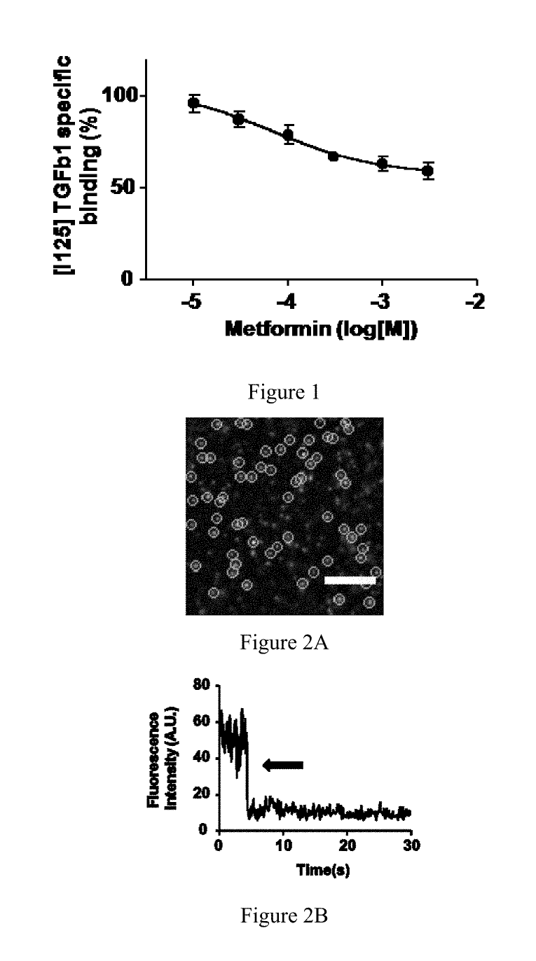

As shown in FIG. 1, Metformin dose dependently inhibited [125I]-TGFβ1 binding to it receptor in 3T3 mouse fibroblasts (log[IC50]=−4.16±0.53).

### Single-Molecule Fluorescence Imaging Analyses TGF-β1-Induced TβRII Dimerization

In the previous study, individual green fluorescent protein (GFP)-tagged TβRII molecules were imaged on the cell membrane by total internal reflection fluorescence microscopy (TIRFM) to study the receptor activation. It has been demonstrated that TβRII exists as monomers at the resting state, and dimerizes upon TGF-β1 stimulation, which supports that the receptor dimerization is essential for receptor activation (Zhang W, Proc Natl Acad Sci U S A 2009; 106:15679-15683).

Single molecule fluorescence imaging was performed with objective-type total internal reflection fluorescence microscopy (TIRFM) using an inverted Olympus IX71 microscope equipped with a total internal reflective fluorescence illuminator'a, 100X/1.45NA Plan Apochromat TIR objective and an intensified CCD (ICCD) camera (Pentamax EEV 512×512 FT, Roper Scientific). Hela cells were transfected with TβRII-GFP plasmid for 4 h, washed, and then imaged in the serum-free and phenol red-free MEM under the fluorescence microscopy. GFP was excited at 488-nm by an argon laser (Melles Griot, Carlsbad, Calif.). Movies of 200-300 frames were acquired for each sample at a frame rate of 10 Hz. For the photobleaching-step counting study, before the single-molecule fluorescence imaging, the cells were treated with TGFβ1(10 ng/ml for 15 min at 37° C.) and different concentrations of metformin, then washed with cold PBS (4° C.) twice and fixed in cold 4% paraformaldehyde/PBS solution for 10 min. To analyze single-molecule fluorescence intensity and the photobleaching steps, regions of interest for bleaching analysis were selected as followed. Firstly, the background fluorescence was subtracted from the movie acquired from the fixed cells using rolling ball method in Image J software (National Institutes of Health). The first five frames of the movie were averaged. Then the image was thresholded (five times of the mean intensity of an area with no fluorescent spots) and filtered with a user-defined program in Matlab (MathWorks Corp) to identify the single molecule spots in the images. Finally, time courses and the integrated fluorescence intensity of regions which were selected according to the method above were extracted for photobleaching analysis. Traces with erratic behavior and ambiguities (30% of traces) were discarded.

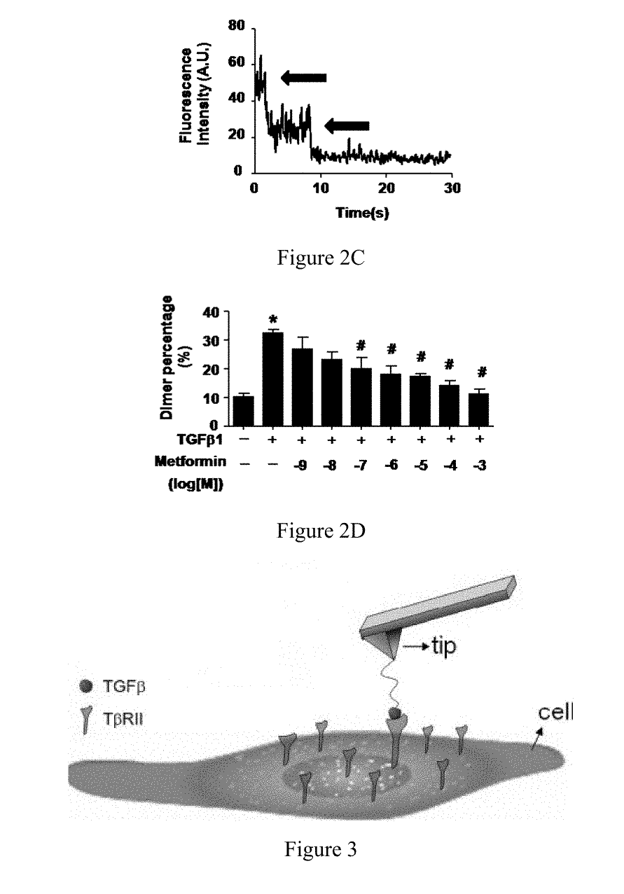

Since TGF-β1-induced TβRII dimerization is the consequence of TGFβ ligandreceptor interaction and is essential for receptor activation ((Zhang W, Proc Natl Acad Sci U S A 2009; 106:15679-15683), the effect of metformin on the formation of ligand induced TβRII dimers were determined by TIRFM. By analyzing the photobleaching traces (FIG. 2A), it was found that 88.8% (778 out of 876 spots from 14 fixed cells) of individual TβRII-GFP molecules were monomers because they bleached in one step (FIG. 2B), 10.7% (94 of 876 spots) of the individual TβRII-GFP molecules were dimers because they bleached in two steps (FIG. 2C), and 0.5% (4 of 876) of the individual TβRII-GFP molecules bleached in three steps. Following the TGF-β1 stimulation, 67.7% (529 of 781 spots) of the individual TβRII-GFP molecules bleached in one step as monomers, 31.6% (247 of 781 spots from nine fixed cells) bleached in two steps as dimers and 0.6% (5 of 781) bleached in three steps. As shown in FIG. 2D, metformin inhibited the percentage of dimers induced by TGF-β1 in a dose-dependent manner.

### Atomic Force Microscopy (AFM) Investigates the Binding Force and the Binding Probability between TGF-β1 and TβRII

The present invention investigated the binding force and the binding probability between TGF-β1 and TβRII on live cells using AFM-based single-molecule force spectroscopy. TGF-β1-modified AFM tips (type: NP-10, Bruker, Santa Barbara, Calif., USA) were prepared as followed. The spring constants of the tips, calibrated by the thermal fluctuation method, were in the range of 0.025-0.045 N/m. The tips were first cleaned and hydroxized through the treatment with chloroform, HF acid, alkaline solution (NH₄OH/H₂O₂/H₂O) 1:1:5, v/v) and piranha solution (98% H₂SO₄/H₂O₂) 7:3, v/v), respectively, to generate Si-OH on the wafers. Then they were transferred to a solution of 1.0% (v/v) MPTMS in toluene, incubated for 2 h at room temperature, and rinsed thoroughly with toluene to be modified with —SH groups. After being dried with N2, the tips were activated by incubation in 1 mg/mL NHS-PEG-MAL, the cross-linker, in dimethyl sulfoxide for 3 h at room temperature, and then rinsed thoroughly with dimethyl sulfoxide to remove any unbound NHS-PEGMAL. The NHS-PEG-MAL was conjugated to the —SH groups on the AFM tips via its MAL end. These activated tips were immersed into a protein (TGF-β1) solution (3×10−8 mol/L in PBS) and incubated at room temperature for 0.5 h. The proteins were bound via their intrinsic amine groups to the NHS end of the PEG derivative. After rinsing with PBS, the protein-modified tips were stored in PBS at 4° C. until use.

Hela cells were transfected with the TβRII-GFP plasmid for 24 h, and the force measurements were performed on a PicoSPM II system with a PicoScan 3000 controller and a large scanner (Agilent, Santa Clara, Calif., USA). The AFM scanner was mounted on an inverted fluorescence microscope (Olympus IX71, Japan). The fluorescent protein-labeled cells were used to guide the AFM tips on the cell expressing TβRII. FIG. 3 shows the schematic diagram of TGF-β1-TβRII binding force measurements which were obtained with TGF-β1-modified AFM tips on HeLa cells. All of the force curves were measured with the contact mode at room temperature using a soft cantilever (0.06 N m-1). The loading rate of the force measurements was 1.0×104 pN/s. The force curves were recorded using PicoScan 5 software (Molecular Imaging, Tempe, Ariz., USA) and analyzed using a program in MATLAB (MathWorks Corp., Natick, Mass., USA.).

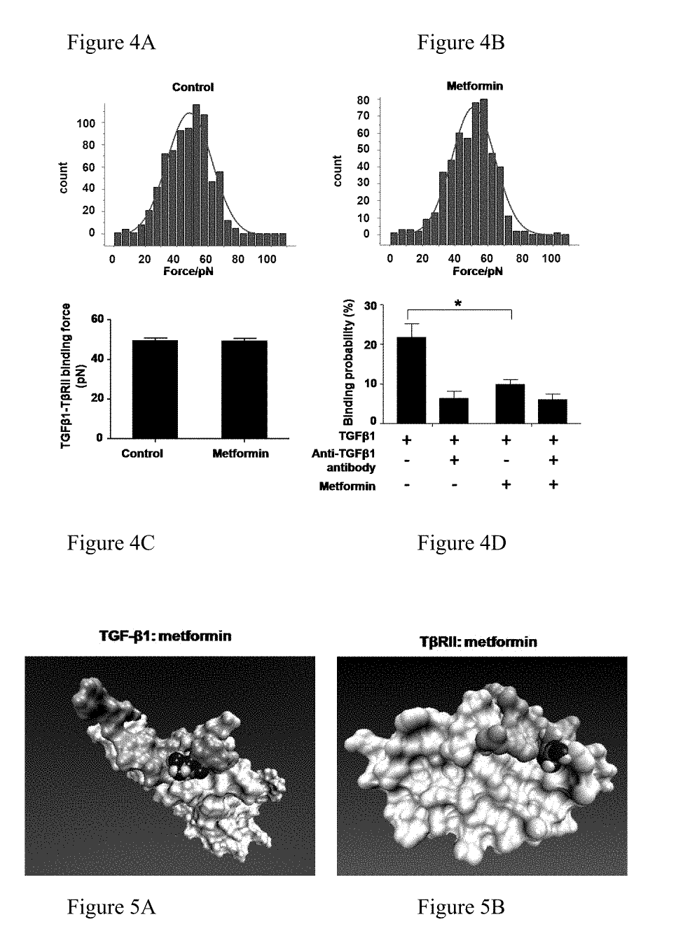

As shown in FIG. 4A and B, the force distribution histogram displayed a single maximum by a Gaussian fit and the binding probability was less than 30%, indicating that single molecule forces were measured. In the cells treated with metformin (50 μM), similar binding forces (measured as the averaged histogram peak value) were observed for TGF-β1 with TβRII on the cell surface as the control (control vs. metformin: 49.5±1.3 vs.49.3±1.4 pN, P>0.05, FIG. 4C). However, metformin decreased the binding probabilities from 21.7±3.5% to 9.9±1.2%, which was similar to the results of the TGF-β1 antibody treatment (6.4±1.9%, FIG. 4D).

### Molecular Docking and Molecular Dynamics Simulation Assess the Potential Binding of Metformin to TGFβ1 and its Receptor

Molecular docking and molecular dynamics (MD) simulation was performed to assess the potential binding of metformin to TGFβ1 and its receptor. The geometry structure of metformin was optimized with Hartree-Forck methods at 6-31+G* level of theory. The crystal structures of TGFβ1 and the extracellular domain of TβRII, were retrieved from the PDB archives (3KFD). Autodock4.2 suite was first applied to predict the preferential binding poses of ligand (metformin) in both TGF-β1 and TβRII. Then the structure of both TGFβ and TβRII bound with metformin were obtained for further evaluation by molecular dynamic simulation. Amber99SB-ILDN forcefield for protein and General Amber force field for ligand was used. The charge parameters of ligand were taken from restrained electrostatic potential calculation. The protein-ligand complex was solvated with TIP3P water. Sodium and Chloride ions were added to neutralize the system. All simulations were carried out with the GROMACS4.6.1 packages and were run in NPT ensemble. The temperature (T=300k) and pressure (p=1 atm) was kept constant using velocity scaling methods and Berendsen barostat methods, respectively. Based on the results of simulation, Molecular Mechanics/Poisson Boltzmann Surface Area methods were used to estimate the binding free energy of metformin on protein.

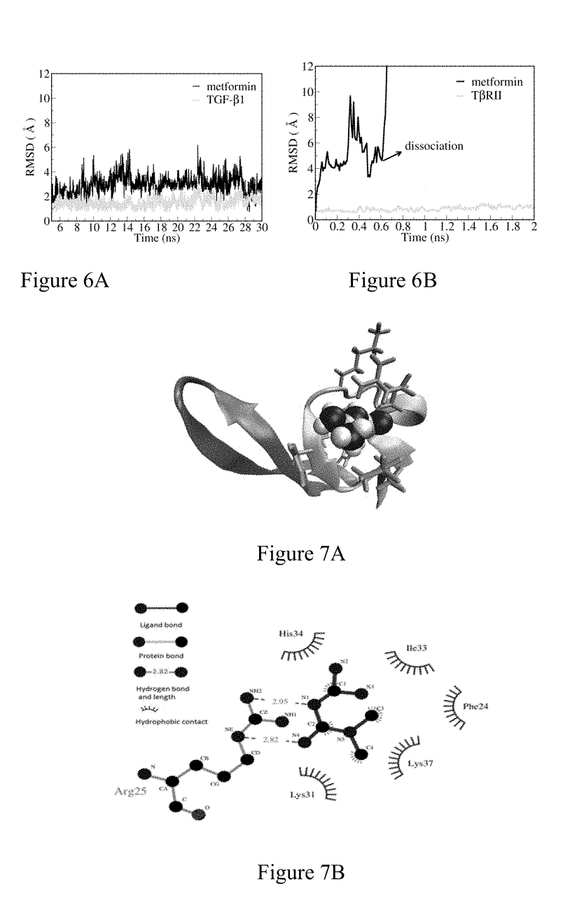

The putative binding site of metformin to TGFβ1 and its receptor TβRII were shown in FIG. 5A and FIG. 5B, respectively. The surface region of TβRII recognized by TGF-β1 is shown in in dark grey. The binding of metformin to TGF-β1 was stable as determined by the root mean square deviation (RMSD) of metformin relative to TGF-β1 (FIG. 6A). However, metformin could not stably bind to the putative binding site of TβRII (extracellular domain). The molecule quickly diffused away from the initial binding site during the molecular simulation (FIG. 6B). Metformin tended to bind in a cave-like structure consisting of the β-strand1 and β-strand2 of TGF-β1 (FIG. 7A). Importantly, this site was partially overlaid with the binding interface of TβRII. The residues in direct contact with TGF-β1 are depicted in FIG. 7B. The binding of metformin was largely attributed to the shape complementarity and hydrogen bond interaction between the guanidine group and Arg25. In addition, the nonpolar components (methyl groups) of metformin were nestled in the hydrophobic bottom of the cave. Thus, the binding stability of metformin to TGF-β1 was further evaluated according to the binding free energy using Molecular Mechanics/Poisson Boltzmann Surface Area methods. The estimated value of the binding free energy (ΔG bind) was −68.50 kJ/mol, which was considered to be sufficiently strong for such a small compound.

### Metformin Binds with TGF-β1 is Determined by Surface Plasmon Resonance (SPR)-Based Assay

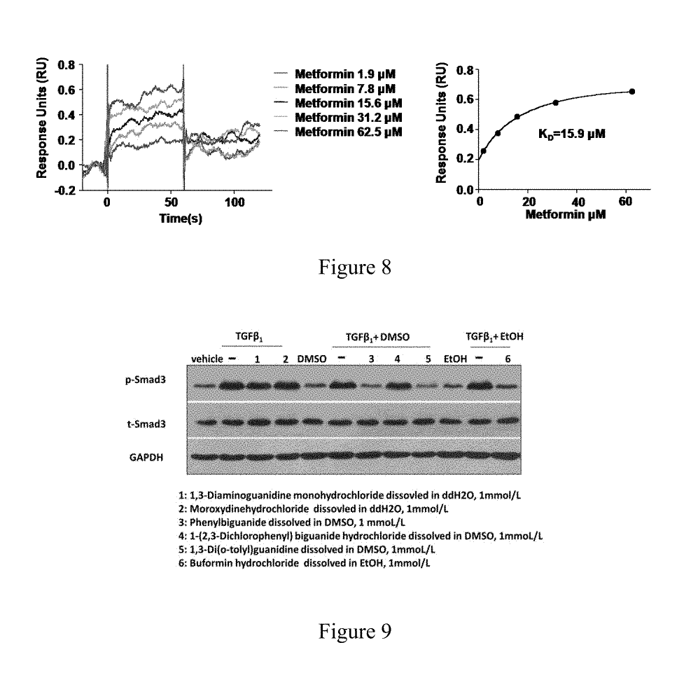

Experiments were performed at 25° C. using a Biacore T200 and the data were analyzed using Biacore T200 evaluation software 2.0 (GE Healthcare, Stockholm, Sweden). TGF-β1 was covalently coupled to a CM5 chip (GE Healthcare) and metformin was injected in a two-fold dilution concentration series ranging from 62.5 μM to 1.9 μM. The steady-state values were calculated from the sensorgrams and plotted against the concentrations. The data were fit to a single site binding model to calculate the K_{D}. Sensorgrams for the binding of metformin and TGF-β1 suggested that the binding increased as the metformin concentration increased (FIG. 8 left panel). And the binding of metformin to TGF-β1 occurred with a K_{D} value of 15.9 μM (FIG. 8 right panel). Therefore, SPR-based assay identified a direct interaction between metformin and TGF-β1.

### Other Guanidine Compounds which have Similar Structure with Metformin Inhibit TGF-β1 Downstream Signaling

To determine if other guanidine compounds which have similar structure with metformin also can be applied as TGF-β1 suppressor, we selected six compounds and detected the effects of these compounds on TGFβ1 induced phosphorylated Smad3 (p-Smad3) using western blotting. The compounds are showed as followed: 1: 1,3-Diaminoguanidine monohydrochloride (dissovled in ddH₂O, 1 mmol/L); 2: Moroxydinehydrochloride (dissovled in ddH2O, 1 mmol/L); 3: Phenylbiguanide (dissolved in DMSO, 1 mmoL/L); 4: 1-(2,3-Dichlorophenyl) biguanide hydrochloride (dissolved in DMSO, 1 mmoL/L); 5: 1,3-Di(o-tolyl)guanidine (dissolved in DMSO, 1mmoL/L); 6: Buformin hydrochloride (dissolved in EtOH, 1 mmol/L). These compounds and TGF-β1 (5 ng/mL) were premixed for 2 h separately and then 3T3 cells were treated with the mixture for 30 min prior to sample collection. Total proteins were extracted by use of RIPA buffer (6.5 mM Tris, pH 7.4, 15 mM NaCl, 1 mM EDTA, 0.1% SDS, 0.25% sodium deoxycholate, 1% NP-40). Bicinchoninic acid reagents were used to measure the protein concentration. Equal amounts of proteins were separated by SDS-PAGE and transferred to polyvinylidene difluoride membranes. The blots were immunoreacted with primary antibodies and secondary antibodies conjugated with horseradish peroxidase. Phospho-Smad3 (Ser423/425) (p-Smad3) and Smad3 were from Cell Signaling Technology (Beverly, Mass., USA). GAPDH antibodies were purchased from Santa Cruz Biotechnology (Santa Cruz, Calif., USA). Protein bands were visualized by enhanced chemiluminescence detection and the intensity was quantified by use of Image-J software.

As shown in FIG. 9, 1,3-Diaminoguanidine monohydrochloride, moroxydinehydrochloride, and 1-(2,3-Dichlorophenyl) biguanide hydrochloride have no effect on p-Smad3 induced by TGFβ1. But other guanidine compounds, such as phenylbiguanide, 1,3-Di(o-tolyl)guanidine, and buformin hydrochloride, attenuate TGFβ1 downstream signaling. This suggested that some guanidine compounds which have similar structure with metformin also inhibit TGF-β1 downstream signaling and can be applied as TGF-β1 suppressor.

1. A composition as a transforming growth factor (TGF)-βsuppressor, the composition comprises an effective amount of guanidine compound or a pharmaceutically acceptable salt thereof, wherein the guanidine compound is selected from the group consisting of metformin, phenylbiguanide, 1,3-Di(o-tolyl)guanidine, and buformin.

2. The composition of claim 1, wherein the pharmaceutically acceptable salt is hydrochloride.

3. The composition of claim 1, further comprising a pharmaceutically acceptable carrier or excipient.

4. The composition of claim 1, wherein the composition can prevent or treat TGF-β associated diseases by suppressing or reducing TGF-β activity, which is a cause of disease.

5. The composition of claim 4, wherein the TGF-β associated diseases comprise fibrosis, cancer, myelodysplastic syndrome, scleroderma, restenosis following coronary artery bypass and angio-plasty, marfan syndrome, or postoperative scarring.

6. The composition of claim 4, wherein guanidine compound or a pharmaceutically acceptable salt thereof is applied as a TGF-β suppressor.

7. A method of treating TGF-β associated diseases, the method comprising administering to the subject guanidine compound or a pharmaceutically acceptable salt thereof, which interacts with TGF-β to block the binding to its receptor and thereafter suppress TGF-β activity to treat TGF-β associated diseases, wherein the guanidine compound is selected from the group consisting of metformin, phenylbiguanide, 1,3-Di(o-tolyl)guanidine, and buformin.

8. The method of claim 7, wherein the pharmaceutically acceptable salt is hydrochloride.

9. The method of claim 7, wherein the TGF-β associated diseases comprise fibrosis, cancer, myelodysplastic syndrome, scleroderma, restenosis following coronary artery bypass and angio-plasty, marfan syndrome, or postoperative scarring.

10. The method of claim 7, wherein the effective amount of metformin compound or a pharmaceutically acceptable salt thereof comprises about 850 mg/day to about 2000 mg/day.
